(19) United States
(12) Patent Application Publication
(10) Pub. No.: US 2006/0224077 A1
(43) Pub. Date: Oct. 5, 2006

(54) Analysis of a skin reactivity and hypersensitivity

(76) Inventors: Gilles Pauly, Nancy (FR); Jean-Luc Contet-Audonneau, Saint-Max (FR); Veronique Gillon, Essey-les-Nancy (FR); Gilles Perie, Nancy (FR); Francois Math, Villers les Nancy (FR); Walter Blondel, Pulnoy (FR); Cedric Pasquier, Nancy (FR)

Correspondence Address: COGNIS CORPORATION;PATENT DEPARTMENT, 300 BROOKSIDE AVENUE, AMBLER, PA, 19002, US

(21) Appl. No.: 10/530,811

(22) PCT Filed: Sep. 27, 2003
(86) PCT No.: PCT/EP03/10766

§ 371 Date: Apr. 5, 2006

(30) Foreign Application Priority Data
Oct. 8, 2002 (FR) 0212462

(51) Int. Cl. A61B 5/04; A61B 5/05

(52) U.S. Cl. 600/544; 600/554; 600/547; 600/546

(57) ABSTRACT

Methods for non-invasive, in vivo determination of the conductivity of nerves in a region of skin and apparatus for carrying out such methods are described, wherein the methods comprise: (a) providing a skin substrate to be analyzed; (b) applying a first non-invasive electrode to a measuring point of the skin substrate; (c) applying a second non-invasive electrode to a second point of the skin substrate; (d) subjecting the skin substrate to stimulation; and (e) analyzing a change in an electrical signal detected by the first and second non-invasive electrodes; wherein the first and second non-invasive electrodes are associated with an evaluation circuit for analyzing the electrical signal detected by the first and second non-invasive electrodes, the evaluation circuit comprising at least one amplifying element, at least one processing element, at least one recording element, and at least one microprocessor.

This invention relates to a process for the non-invasive in vivo characterization and analysis of the reactivity and/or oversensitivity of a zone of the skin at face level or optionally appendages of the skin or optionally the scalp by determining the conductivity of the nerves in that region.

The skin is equipped with a particularly effective communication and control system of which the function is to protect the organism from the environment. This system contains a very dense system of highly specialized, outgoing autonomous nerve branches and sensory, feeder nerve branches which is distributed over all layers of the skin. The information passing through this network is processed in the central nervous system and can produce an inflammatory reaction through the antidromic propagation of impulses. The response activity of a nerve can be determined from the neuropeptides released and the receptors of the corresponding target structures. The innervation of the skin reaches the uppermost layers of the epidermis. The skin nerves contain only sensory or autonomous nerve fibers which can be provided with a myelin sheath. In the sensory region of the skin, the fibers provided with a myelin sheath can be classified by diameter and conductivity rate into rapidly conducting groups Aδ and Aβ while the fibers of type C with no myelin sheath form the other group.

According to the publication by Metze, D. and Luger, T.: Nervous System in the Skin in “The Biology of Skin”, Freinkel, R. K. ed.; Woodley, D. T., ed., pages 153-176, several neurophysiological tests have shown that the Aβ fibers transmit tactile sensitivity while the Aδ and C fibers transmit contact, temperature, unpleasant sensations, itching and various other physical and chemical stimuli.

Before the stimulus, a neuron has a slightly negative electrical polarity. The action potentials are electrical signals which are controlled by the presence and concentration of ions around the nerve cell and which spread out along it. These potentials have various phases:
a quiescent state in which the potential of the membrane is at rest (before release of the potential of the nerve action),
a depolarization state which is encountered in the event of strong inversion of the membrane potential,
a repolarization state which occurs when the membrane potential returns to the quiescent state,
the relaxation state when the membrane potential is back in the quiescent state.

According to numerous scientific works, an increasing number of people have oversensitive skin, particularly at face level, with unwanted reactions to the use of cosmetic and toilet products and to climatic variations. According to the article by Willis, S. M.; Shaw, S.; de la Charrière, O.; Baverel, M., Reiche, L., Jourdain, R.; Bastien, P., Wilkinson, J. D.: Sensitive Skin: An Epidemiological Study, in British Journal of Dermatology, 2001, No. 145, pages 258-263, researchers have conducted an epidemiological study among the British population in order to evaluate the frequency of dermal oversensitivity and its harmful effects in conjunction with cosmetic products and to determine the factors which can be related to dermal oversensitivity. 51.4% of women and 38.2% of men said their skin was oversensitive. Among the female population, unpleasant subjective symptoms of dermal sensitivity to cosmetic products (burning, stinging, itching . . . ) occurred more frequently in people who regarded themselves as oversensitive (53%) than in people who regarded themselves as non-sensitive (17%).

According to the publication by de la Charrière, O.: La peau—une réalité clinique. Peau et système nerveux, May 2000, St. Etienne, France, a distinction was drawn between two main types of oversensitivity: sensitivity to environmental factors and sensitivity to cosmetics, these main types being particularly sensitive to capsaicin. This specific feature suggests that the innervation of the skin is a key factor in the physiology of dermal oversensitivity.

According to the article by Wallengreen, J.; Hakanson, R.: Effects of capsaicin, bradykinin and prostaglandin E2 in the human skin, in British Journal of Dermatology, 1992, No. 126, pages 111-117, capsaicin is the stimulating active principle of chilli peppers and is capable of exciting the C fibers and releasing tackykinins, such as the substance P, and a special peptide, CGRP (Calcitonine Gene Related Peptide) which, in the initial applications, is responsible for the sensation of temporary pain, burning and itching, and thereby relieving the sensory neurons of the skin of substance P.

Itching is one of the most commonly described symptoms of dermal oversensitivity. It may be defined as an unpleasant sensation of the skin which initiates the desire to scratch. Itching is produced by a certain number of chemical compounds, although it can also be produced by moderate thermal, electrical or mechanical stimuli. It may be regarded as a nociceptive sensation.

According to the article by Schmeiz, M.; Schmidt, R.; Nickel, A.; Handwerker, H. O.; Torebjork, H. E.: Specific C-receptors for itch in human skin, in J. Neurosci., 1997; 17; 8003-8, micrographs (invasive microelectrodes) have clearly shown that itching is transmitted by a sub-population of nociceptive polymodal neurons of the C type with no myelin sheath. The ends of these neurons are free nerve ends which are present both at dermis and at epidermis level.

Accordingly, the relationship between the skin and the nervous system is presently one of the most studied areas of dermal biology.

According to the article by Misery, L.: Les nerfs à fleur de peau, in International Journal of Cosmetic Science, 2002, No. 24, pages 111-116, new research fields have now been opened up: neurodermatology and neurocosmetics which are said to modulate the neuro-immune system of the skin (NICS). According to the article by Lauria, G.; Holland, N.; Hauer, P.; Cornblath, D. R.; Griffin, J. W.; McArthur, J. C.: Epidermal innervation, changes with aging, topographic location and in sensory neuropathy, in Journal of Neurological Sciences, 1999, No. 164, pages 172-178, intra-epidermal nerve fibers were observed by chemical—histological—immunological coloration. The main biopsy was used for the purpose of analyzing the terminal regions of small nerve fibers, more particularly to determine the features of type C nerve fibers with no myelin sheath and small, type Aδ nerve fibers with a myelin sheath. However, this method is invasive and highly traumatizing and is therefore unsuitable for research in the cosmetics field.

The face is the most important location for dermal oversensitivity. In the face, skin innervation is taken care of by the branches of the trigeminal nerve. The trigeminal nerve consists of three main branches, the ophthalmic branch (V1, sensory), the maxillary branch (V2, sensory) and the mandibular branch (V3; motor and sensory).

Our investigations were particularly concerned with the maxillary branch.

According to the article by Hindy, A. M. and Raouf, F. A.: A Study of infraorbital foramen, canal an nerve in adult egyptians, in Egyptian Dental Journal, 1993, No. 39, pages 573-580, the maxillary branch of the trigeminal nerve penetrates through the infraorbital foramen into the skull. Its sensory branches innervate the face, the cheeks and the teeth of the maxilla. The location of the foramen differs considerably from one individual to another which leads to difficulties in attempts to record the conductivity of the trigeminal nerve because the position of the foramen has to be determined beforehand. The potential of the objectivized nerve may advantageously be investigated at the same time as, and related to, the brain potentials.

In the article by Agostino, R.; Cruccu, G.; lannetti, G. D.; Innocenti, P., Romaniello, A.; Truini, A.; Manfredi, M.: Trigeminal small-fibre dysfunction in patients with diabetes mellitus: a study with laser evoked potentials and corneal reflex, in Clinical Neurophysiology, 2000, No. 111, pages 2264-2267, the function of the small fibers of the mandibular nerve was studied in the special case of diabetics—more particularly the potentials stimulated after laser stimulation of the part of the skin on the edge of the lower lip. Using this method, it was shown that a potential produced after laser stimulation has a longer average latent time and a lower amplitude in diabetics than in non-diabetics, which is indicative of functional disturbances of small adjoining fibers of the mandibular nerve.

An entirely new study has shown that the feeling of an electrical stimulation of sensitive skin was increased in relation to non-sensitive skin (Yokota, T.; Matsumoto, M.; Sakamaki, T.; Hikima, R.; Hayashi, S.; Yanagisawa, M.; Kuwahara, H.; Agawa, T.; Hayase, M.: Classification of sensitive skin and development of a treatment system appropriate for each group, 22nd IFSCC Congress, Edinburgh, 2002).

According to the above-mentioned article by Johansson, R. S.; Trulsson, M.; Olsson, K. A., Westberg, K. G.: Mechanoreceptor activity from the human face and oral mucosa, in Experimental Brain Research, 1988, No. 72, pages 204-208, techniques from the field of microneurography were commonly used to investigate the innervation of the face. These techniques comprise piercing the nerve to be studied with microelectrodes of tungsten at the place where it emerges from the infraorbital foramen and recording the signals at the level of the nerve fiber. Unfortunately, this technique has the disadvantage that it is invasive and, hence, unsuitable for a field such as cosmetics.

According to the publication by Rau, G.: Measuring sensor for the non-invasive detection of electro-physiological quantities, U.S. Pat. No. 4,685,466, a measuring sensor provided with an electrode having a fine, needle-like part for the detection of local conductivity is proposed for the non-invasive detection of neurophysiological electrical signals at skin level. However, the penetration of at least one electrode into the Stratum corneum is mentioned in this publication. Although the scope of application of such a detector is not explained, it is clear that the desired objective is not recording the conductivity of nerves.

Today, the conductivity of nerves in human beings can be non-invasively investigated by recording the potentials of the nerves starting from most of the surface nerves of the body—forearm, arm, leg—using surface electrodes. However, these investigations have not yet been adapted to the facial skin.

The problem addressed by the present invention was to fill that gap.

The present invention relates to a process for the non-invasive in vivo characterization and analysis of the reactivity and/or hypersensitivity of a skin zone in the face or optionally appendages of the skin or optionally the scalp by determining the conductivity of the nerves in that region.

The present invention also relates to an apparatus which enables this process to be applied and to its use in the cosmetics field.

The process according to the invention which uses the electrogenesis of the nerves and/or the brain is characterized in that:
two non-invasive electrodes (1,1′) are applied, at least one of the two electrodes being applied to a specific point of the zone of the skin to be analyzed, the measuring point, in order to detect the electrical activity of the nerves of the skin and/or the subcutaneous nerves at that point or even the electrical activity of the brain. These electrodes are associated with a circuit for evaluating the signals detected by the electrodes, this evaluation circuit comprising amplifying elements (2, 2′, 3), processing elements (7) and recording elements for the signals and also a microprocessor (8),
the skin zone to be analyzed is exposed to a stimulation, more particularly an electrical stimulation, and
the changes in the electrical signals detected by the electrodes driven by the stimulation as a function of time are analyzed over a certain period.

The connection thus makes it possible simultaneously to analyze and relate to one another
the responses at brain level, for example the brain potentials produced, but also by any imaging technique and/or by biophysical processes and/or by description of the sensations
and the nerve potentials which reveal nerve activity.

The signals detected by the measuring electrodes are usually electrical signals. However, it is pointed out that the electrical activities of the skin or brain could also be analyzed using the magnetic field generated without departing from the scope of the invention.

According to the invention, the reactivity and/or hypersensitivity of the skin and/or its appendages and/or the scalp can be caused by any change and/or pathology affecting the central and/or peripheral nervous system and/or the skin and its appendages, such as those of immunological origin and/or metabolism- and/or vessel-induced, hyperaesthetic or hypoaesthetic causes and inflammations, dry skin or irritation of any kind. This reactivity and/or hypersensitivity can correspond to such symptoms as itching, flushing, irritation, heat, stinging, burning . . . produced by activity of the nerve fibers and/or the release of substances, for example of neurological and/or skin-related origin. These various symptoms can occur after stress or, for example, physical attacks (more particularly electrical or mechanical or thermal or light-induced or vibration or electromagnetic influences) and also after chemical, physiological or biological attacks.

According to another aspect of the invention, the skin zone to be analyzed is subjected to a stress, for example a chemical or a physical stress, more particularly electrical, mechanical, electromagnetic or thermal in character, light waves or vibrations, or even to a neurological or psychic stress, and the changes as a function of time in the signals detected by the electrodes influenced by the stimulation—with and without stress—are compared. These various stress types can readily be routinely produced.

According to another aspect of the invention, the measuring electrode is so positioned that it transmits to the evaluation circuit signals that are representative of the activity of one of the branches of the trigeminal nerve.

The invention enables the electrical activity of the maxillary branch of the trigeminal nerve to be analyzed in a particularly advantageous manner. The electrical activities of the ophthalmic and/or mandibular branches of this nerve can also be analyzed without departing from the scope of the invention.

According to another aspect of the invention, at least two measuring electrodes are applied to the skin zone to be analyzed, at least one of these measuring electrodes being so designed that it can simultaneously measure the impedance of the skin, and a weak alternating current is applied to at least a first electrode so that the impedance of the skin is measured at the associated measuring point. This weak alternating current can be transmitted, for example, by an electrical alternating current field.

In order to enable the process according to the invention to be carried out satisfactorily, this weak alternating current and its effects must of course be absent during the measurement.

According to another aspect of the invention, the measuring electrode(s) is/are positioned in dependence upon the impedance value of the skin. This positioning can also be carried out in other ways without departing from the scope of the invention, more particularly by imaging and/or biophysical techniques.

In another embodiment of the invention, the electronic system (2,2′) can increase the potential difference between the electrodes 1 and 1′without transmitting the common-mode signal.

Accordingly, the invention also solves the problem of the in vivo location of the infraorbital foramen.

The present invention also relates to an apparatus for carrying out the process described above. According to the invention, this apparatus is characterized in that it comprises:
at least one non-invasive electrode which is suitable for detecting the signals representative of the electrical activity of the nerves of the skin and/or the subcutaneous nerves at the level of a specific zone of the skin to be analyzed or even the electrical activity of the brain,
at least one stimulation electrode associated with an electrical stimulator,
a non-invasive reference electrode and
a circuit for evaluating the signals detected or transmitted by the electrodes which comprises amplifying elements, processing elements and elements for recording those signals and a microprocessor, so that curves representative of the changes as a function of time in the signals detected by the measuring electrode after a stimulation can be created and displayed.

The apparatus according to the invention comprises in particular a computer equipped with calculating means which enables the relationship between the stimulation and the recording to be analyzed, more particularly with specific, adapted processing of these signals.

In view of the fact that the apparatus according to the invention is intended for use on a human subject, it is essential, more particularly on safety grounds, that all parts in contact with this subject are completely insulated from the mains current which can be achieved by various systems, more particularly optical, mechanical, acoustic, capacitive, magnetic or hertzian systems.

According to the invention, the measuring electrode and optionally the reference electrode are non-polarizable or substantially non-polarizable and are made in particular from stainless steel, from tungsten or from a noble metal, such as Au or Ag/AgCl.

In order to improve contact between these electrodes and the skin of the subject, to promote the conductivity of the nerve potential and to avoid spurious signals as far as possible, a suitable liquid or an electrically conductive gel, for example water containing sodium chloride, may be applied to the skin.

In another embodiment of the invention, the measuring electrode is mounted on the end of a hinged arm which is held on the head of the subject by a suitable holder, more particularly a helmet. The presence of such a holder is suitable for facilitating the positioning of the specific measuring points of the skin and for holding the measuring electrode in that position irrespective of the head movements of the subject.

In a preferred embodiment of the invention, the apparatus comprises at least two measuring electrodes of which at least one is so designed that it can simultaneously measure the impedance of the skin.

The first measuring electrode may advantageously co-operate with at least one generator of a variable voltage which is associated with at least one transmitting aerial designed to be erected in its vicinity, so that it enables the skin impedance to be measured.

According to the invention, the processing elements usually consist of one or more filters or one or more analog/digital converters.

In another preferred embodiment of the invention, the amplifying elements comprise at least one preamplifying module for the signals detected by the measuring electrode(s) which consists of at least one preamplifier with a gain of 1 or less, of which the input impedance is high over a broad voltage range of at least + or −3 volts, and on the other hand an amplifying module for the preamplified measuring signals detected by the measuring electrode(s), more particularly consisting of a measuring amplifier with a variable or fixed gain, preferably with values of 100 to 1,000.

The amplifying elements are directly connected to the electrode which is located in a zone to be determined.

During the electrical stimulation, a voltage of several volts appears at the level of the measuring electrodes. However, this voltage must not produce a current which would run the risk of polarizing the electrodes. For this reason, it is desirable to equip the apparatus according to the invention with a preamplifying module consisting of at least one low-gain preamplifier.

According to the invention, the preamplifying module has to be kept as free as possible from spurious signals and from the impedance of the cables by which it can be connected to the measuring electrode(s). To this end, the preamplifier may be arranged in the immediate vicinity of the associated measuring electrode. Another possibility is to connect the measuring electrode(s) to the input of the associated preamplifier via a shielded cable. In this variant of the invention, the shield(s) of the shielded cable(s) are preferably connected to the output(s) of the following preamplifier which may be any one of various types, namely:
a servo amplifier with a gain of 1,
a non-servo controlled amplifier with a gain of 1, for example with a two-pole transistor with a common collector or a field-effect transistor with a common drain,
an amplifier with a gain of x with a following attenuator in a ratio of x,
a voltage which is tapped at a particular point of an amplifier and which is capable of having a value that comes very close to the input voltage, the shield tension 17 (17′) having to follow the wire tension 18 (18′), this input voltage optionally being applied to a following amplifier, or
a voltage which is tapped at a particular point of a two-channel amplifier and which is capable of having a value that comes very close to the average of the input voltages, this voltage optionally being applied to a following amplifier.

The present invention also relates to the application of the process and the apparatus described above in the cosmetics field. In addition, the activity of the muscles, for example, can be determined by electrical stimulation. In this field, the invention provides not only for analysis of the reactivity and/or hypersensitivity of the skin or its appendages or even the scalp, but also for their treatment. More particularly, the invention provides for the characterization and treatment of the sensitive, sensitized, hyperreactive, stimulated, stressed skin and scalp, for the care of the skin after exposure to the sun or to lasers or after epilation or even for the treatment of the scalp which tends to develop serious seborrhoea and/or dandruff and/or alopecia. This treatment may be carried out, for example, with such compounds as dermocorticoids, local anaesthesia, compounds or mixtures of compounds with pain-relieving, inflammation-inhibiting and/or UV-protecting and/or calming and/or soothing and/or moisturizing properties and/or properties against sensitivity of the skin and/or agonists or antagonists of the neuropeptides, either individually or in combination with one another. For example, the following substances and derivatives may be used: stilbene, resveratrol, rhapontin, panthenol, allantoin, bisabolol, Karite butter, glycyrrhetic acid, chicory acid, zinc salts, coffee esters, phenolic acid esters, thermal waters, glycerin, calamine, azulene oil, peptides, plant extracts, more particularly from plants of the families Sapotaceae (argania, butyrospermum), Linaceae (linum), Asteraceae (cichorium, calendula, arctium), Gramineae (avena), Hamamelidaceae (hamamelis), Rosaceae (prunus, filipendula), Asphodelaceae (aloe), passifloraceae (passiflora), Nymphaceae (nymphea), Araliaceae (hedera), Clusiaceae (calophyllam), Malvaceae (althea), Leguminosae (eperua, vigna), Anacardiaceae (spondias), Caesalpiniaceae (cassia), Bombaceae (adansonia), Musaceae (musa), Papaveraceae (papaver), Solanaceae (solanum, capsicum), Menispermaceae (curarea), Loganiaceae (strychnos), Apocynaceae (rauvoffla), Rubiaceae (cinchona), Cannabinaceae (cannabis), Ericaceae (arctostaphylons, rhododendron). For example, products from the Cognis Group with the following names and compositions may also be used: Anasensyl LS 9322 (INCl: mannitol, ammonium glycyrrhizate, caffeine, zinc gluconate, Aesculus hippocastanum extract) Biophytex LS 8740 (INCl: propylene glucol, Ruscus aculeatus root extract, Centella asiatica extract, panthenol, water, Calendula officinalis flower extract, hydrolyzed yeast proteins, Aesculus hippocastanum extract and ammonium glycyrrhizate) Rhizodermin LS 6277 (INCl: water, propylene glycol, Symphytum officinale extract, Arctium majus root extract, ammonium glycyrrhizate and allantoin), Cytokinol LS 9028 (INCl: hydrolyzed casein, hydrolyzed yeast proteins and lysine HCl) Fructinase AS LS 9650, Fructiplex SR LS9651, Mellidyn LS 9657, Eperuline PW LS 962, generol R, S, oligochitosan.

The features of the process and the apparatus according to the invention are described in more detail in the following with reference to the accompanying drawings, wherein:

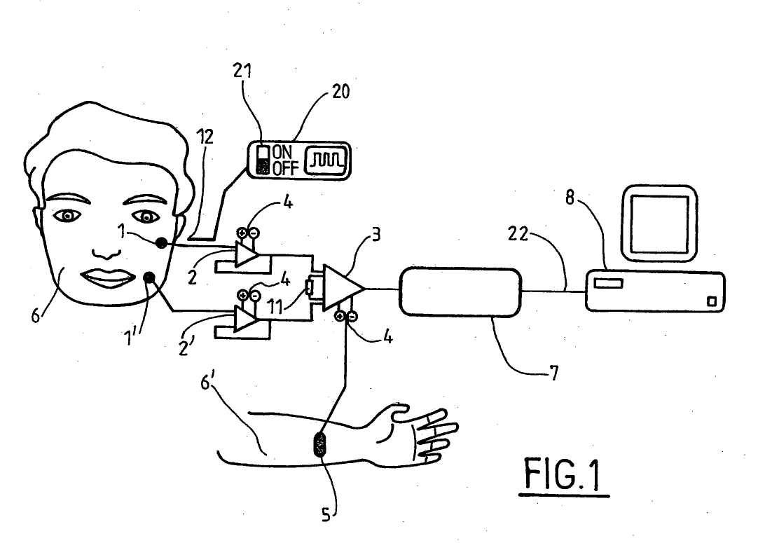

FIG. 1 schematically illustrates the apparatus.

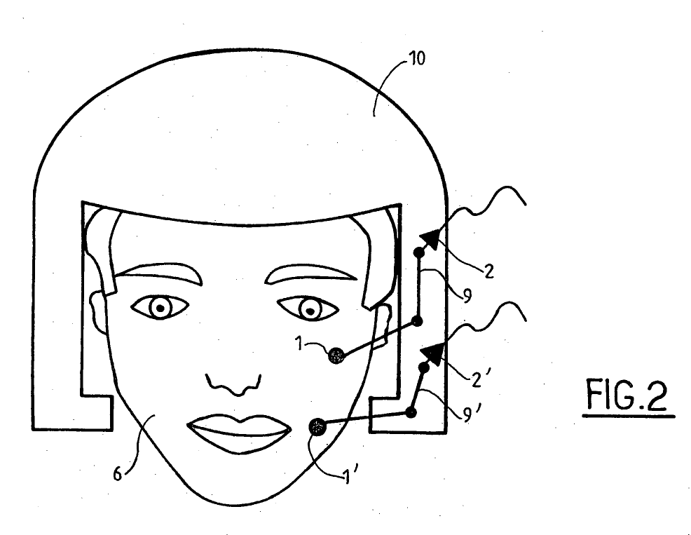

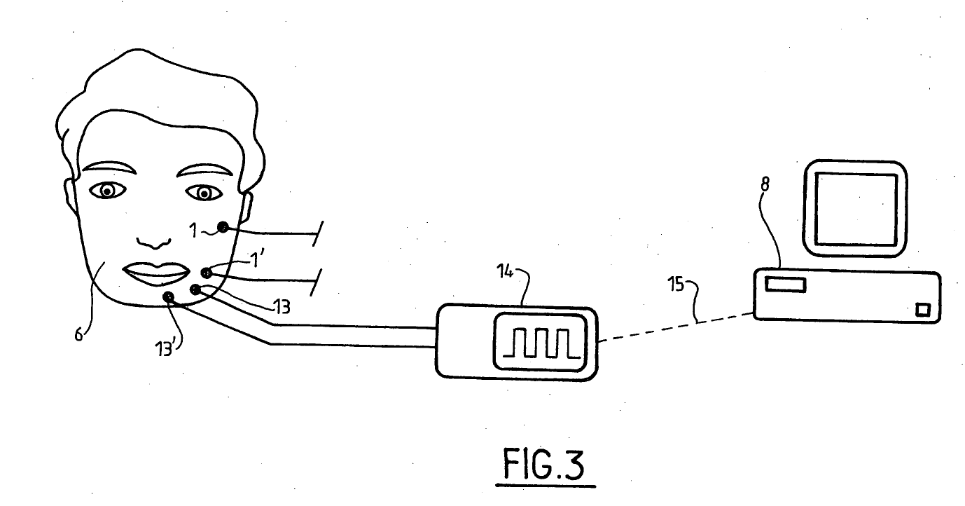

FIGS. 2 and 3 show details of FIG. 1.

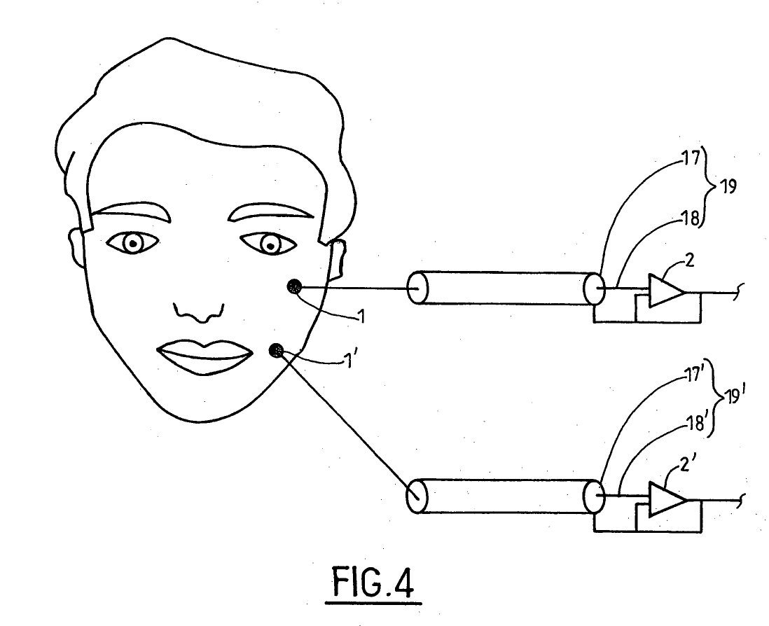

FIG. 4 shows a variant of the apparatus.

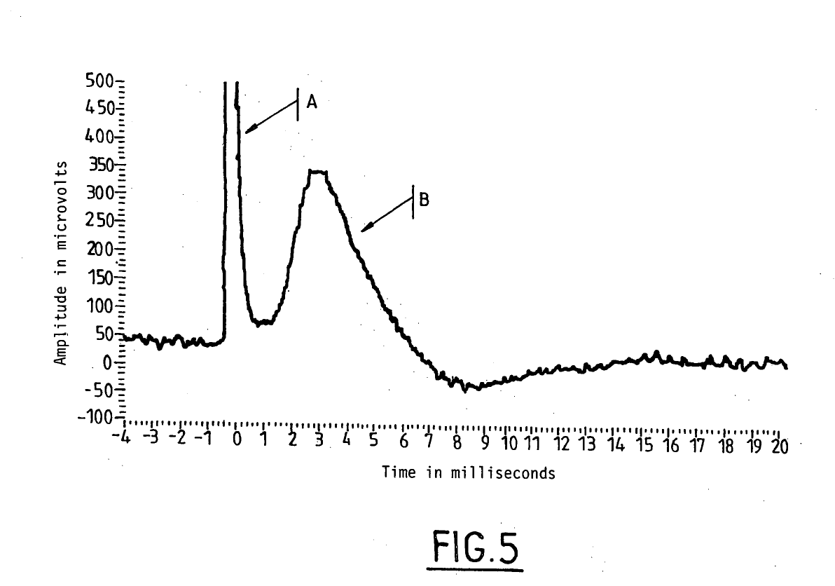

FIG. 5 is a curve showing the changes in the amplitude of the potential of the Aβ fibers after a positive electrical stimulation as a function of time.

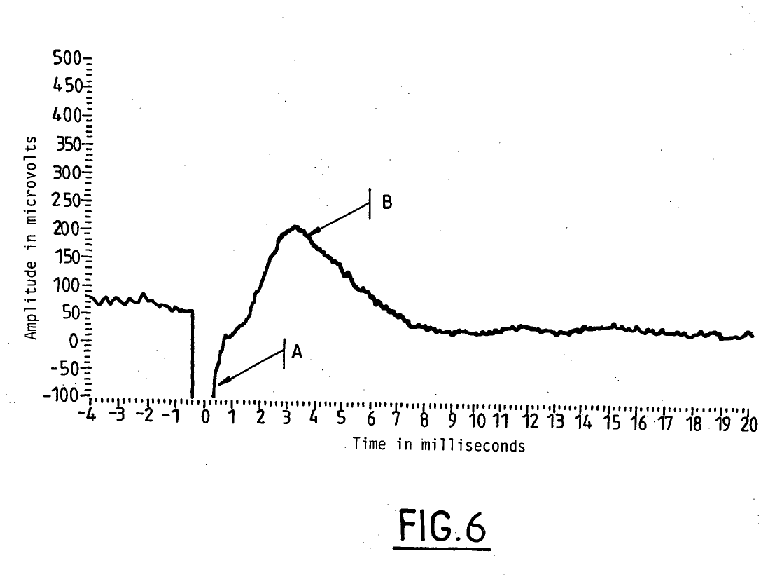

FIG. 6 is a curve which corresponds to FIG. 5, but after a negative electrical stimulation.

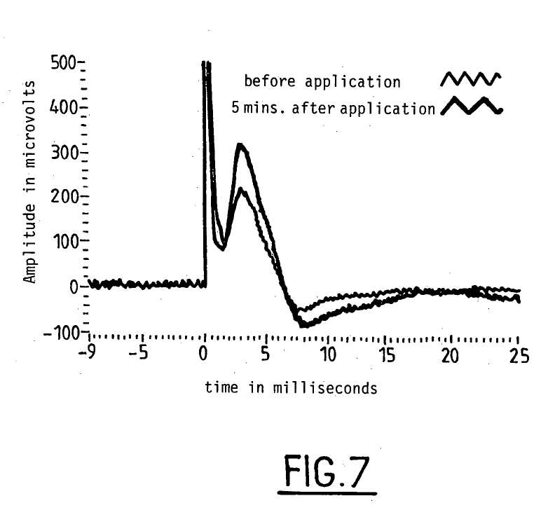

FIG. 7 shows the development of the action potential of the maxillary nerve 5 minutes after the application of capsaicin.

As shown in FIGS. 1, 2 and 3, the apparatus comprises two non-invasive measuring electrodes 1, 1′ of Au and/or Ag/AgCl which are brought into intimate contact with the skin of a subject at face level 6. Accordingly, the measuring electrodes 1, 1′ can detect signals that are representative of the electrical activity of the sensory nerves of the skin and/or the sensory subcutaneous nerves at that level. One of the measuring electrodes 1 is so designed that it simultaneously records the impedance of the skin.

Referring to FIG. 1, the measuring electrodes 1, 1′ co-operate with a non-invasive reference electrode 5 which is in intimate contact with the skin of the subject at forearm level 6′. The measuring electrodes 1, 1′ and the reference electrode 5 are connected to a circuit for evaluating the signals they transmit which is described in more detail in the following. This evaluation circuit essentially comprises a microprocessor 8 which enables the curves representative of the change as a function of time in the signals detected by the electrodes 1, 1′ and 5 to be created and displayed.

As shown in FIG. 3, the apparatus also comprises two stimulation electrodes 13, 13′ which are also located at face level 6 on the subject. These stimulation electrodes 13, 13′ are connected to an electrical stimulator 14 which is connected to the microprocessor 8 by insulated connecting elements 15.

As shown in FIG. 1, each of the electrodes 1, 1′ is connected to a low-gain preamplifier 2, 2′. These preamplifiers 2, 2′, which enable the impedance to be adapted, may be of the TL 082 type for example. The output of the preamplifiers 2, 2′ is connected to a measuring amplifier 3 of which the frame is directly or indirectly connected to the reference electrode 5. The measuring amplifier 3 may advantageously be an amplifier of the AD 620 type (Analog Device, USA), of which the variable gain is predetermined by the value of an associated resistance 11. The preferred gains of the measuring amplifier 3 are between 100 and 1,000 which corresponds to a resistance 11 of 50 to 500 ohms.

As can be seen in FIG. 1, the measuring amplifier 3 and the two preamplifiers 2 and 2′ are fed by batteries 4, so that thorough isolation of the face 6 or the forearm 6′ of the subject is guaranteed.

The evaluation circuit further comprises an insulated medical analog/digital converter 7 of the type used in electroencephalography or electrocardiography. The converter 7 converts the analog signal it receives from the measuring amplifier 3 into digital information which in turn is transmitted by an adapted cable 22 to the microprocessor 8.

As shown in FIG. 1, the apparatus also comprises elements by which the measuring electrodes 1, 1′ can be positioned according to the variations in the impedance of the skin. These elements consist of a generator (1 kHz to 1 MHz) 20 and a transmitting aerial 12 located in the vicinity of the measuring electrode 1. A contact breaker, for example in the form of a switch 21, enables the generator 20 to be switched off during the measurement.

As shown in FIG. 2, the measuring electrodes 1, 1′ are mounted at the end of hinged arms 9, 9′ which are held on the head of the subject by a helmet 10. The preamplifiers 2, 2′ are arranged in the immediate vicinity of the hinged arms 9, 9′ and the measuring electrodes 1, 1′ in order to reduce their susceptibility to failure.

In a modified embodiment shown in FIG. 4, which also enables interference effects to be reduced, the preamplifiers 2, 2′ are connected to the measuring electrodes 1, 1′ by cables 19, 19′ which consist of wires 18, 18′ and a shield 17, 17′. The shield 17, 17′ is connected to the output of the associated servo amplifier 2, 2′.

As shown in FIGS. 5 and 6, the computer 8 is equipped with software suitable for processing the digital signals transmitted to it, so that it can create and display the curves representing the variations in the amplitude of the nerve potential as a function of time.

FIG. 5 shows the variations in the amplitude of the potential of the fibers Aβ in microvolts as a function of the time in milliseconds after a positive electrical stimulation. FIG. 6 shows the same variations after a negative electrical stimulation. In these Figs., the peaks A and B each correspond to an artefact produced by the stimulation and by the potential of the nerve activity of the fibers Aβ.

FIG. 7 compares the variations in the action potential of the maxillary nerve (V2, sensory) in response to an electrical stimulation at face level before and after the application of capsaicin.

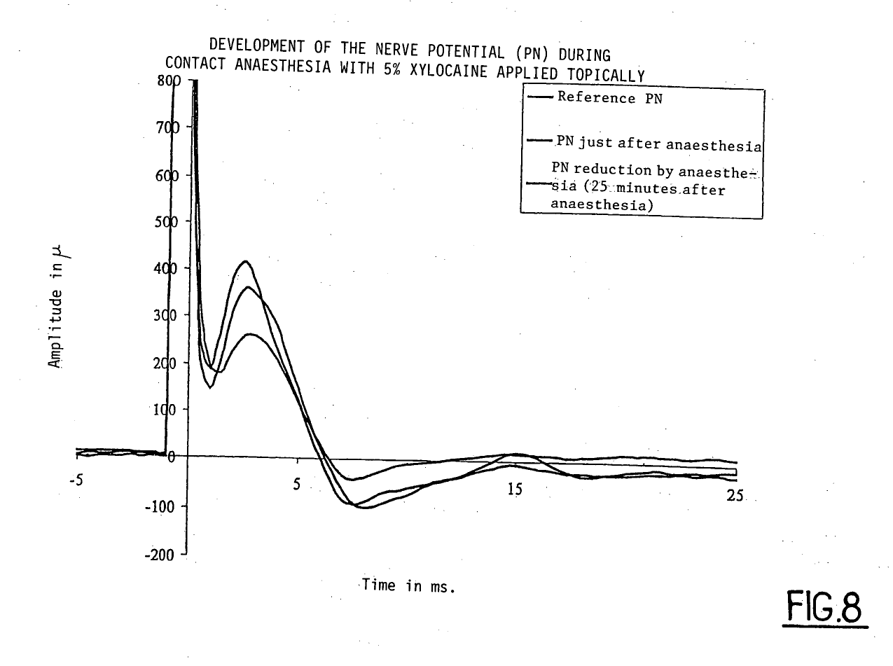

FIG. 8 compares the variations in the action potential of the maxillary nerve (V2, sensory) in response to an electrical stimulation at face level before and immediately after the topical application of xylocaine (5%). The potential of the nerve was observed during the phase of re-finding the sensations. A cream containing 0.75% capsaicin was used for this purpose and was applied in a quantity of 2 mg/cm² to a 2×4 mm skin zone immediately above the upper lip.

The action potential of the nerve which was directly recorded in vivo by the non-invasive process according to the invention was changed after the application of capsaicin. After application, the amplitude of the nerve potential had increased by 75% which signifies an increase in the electrical activity of the nerve.

1-18. (canceled)

19. A method for non-invasive, in vivo determination of the conductivity of nerves in a region of skin, said method comprising:
(a) providing a skin substrate to be analyzed;
(b) applying a first non-invasive electrode to a measuring point of the skin substrate;
(c) applying a second non-invasive electrode to a second point of the skin substrate;
(d) subjecting the skin substrate to stimulation; and
(e) analyzing a change in an electrical signal detected by the first and second non-invasive electrodes;
wherein the first and second non-invasive electrodes are associated with an evaluation circuit for analyzing the electrical signal detected by the first and second non-invasive electrodes, the evaluation circuit comprising at least one amplifying element, at least one processing element, at least one recording element, and at least one microprocessor.

20. The method according to claim 19, wherein the stimulation comprises electrical stimulation.

21. The method according to claim 20, wherein the electrical stimulation is provided by a stimulation circuit comprising at least two stimulation electrodes in contact with an area of the skin substrate subject to the stimulation and an electrical stimulator connected to the microprocessor.

22. The method according to claim 19, wherein the skin substrate subjected to stimulation is further subjected to a stress and an electrical signal detected by the first and second non-invasive electrodes with the stress is compared to the electrical signal detected by the first and second non-invasive electrodes without the stress.

23. The method according to claim 19, wherein the skin substrate comprises facial skin.

24. The method according to claim 19, wherein the first non-invasive electrode is positioned such that it is capable of transmitting signals representative of the electrical activity of at least one branch of a facial trigeminal nerve selected from the group consisting of an ophthalmic branch, a maxillary branch, a mandibular branch and combination thereof.

25. The method according to claim 24, wherein the at least one branch comprises the maxillary branch.

26. The method according to claim 19, further comprising applying a weak alternating current to the first non-invasive electrode and measuring the impedance of the skin substrate.

27. An apparatus for non-invasive, in vivo determination of the conductivity of nerves in a region of skin, said apparatus comprising:
(a) at least one non-invasive measuring electrode suitable for detecting a signal representative of the electrical activity of (i) a sensory nerve of a skin substrate or of (ii) the brain;
(b) an electronic stimulator connected to at least one stimulation electrode;
(c) at least one reference electrode;
(d) a circuit connected to the at least one non-invasive measuring electrode, the electronic stimulator, and the at least one reference electrode for evaluating signals detected or transmitted by said electrodes, the circuit comprising at least one amplifying element, at least one processing element, at least one recording element, and at least one microprocessor; such that a curve representative of change in the signal detected by the at least one non-invasive measuring electrode after a stimulation, as a function of time, can be created and displayed.

28. The apparatus according to claim 27, wherein the at least one non-invasive measuring electrode is non-polarizable or weakly polarizable.

29. The apparatus according to claim 27, wherein the at least one non-invasive measuring electrode comprises a material selected from the group consisting of stainless steel, tungsten, noble metals and mixtures thereof.

30. The apparatus according to claim 27, further comprising an adaptable holder and an adjustable arm having a first end and a second end, wherein the first end is connected to the adaptable holder, and wherein the at least one non-invasive measuring electrode is connected to the second end.

31. The apparatus according to claim 27, comprising at least two non-invasive measuring electrodes, wherein at least one non-invasive measuring electrode is capable of measuring impedance of the skin substrate.

32. The apparatus according to claim 31, further comprising at least one adjustable voltage generator associated with at least one transmitting aerial erected in proximity to the at least one non-invasive measuring electrode capable of measuring impedance.

33. The apparatus according to claim 27, wherein the at least one amplifying element comprises at least one preamplifier having a high input impedance over a voltage range of from −3 to +3 volts.

34. The apparatus according to claim 33, wherein the at least one preamplifier is connected directly to the at least one reference electrode.

35. The apparatus according to claim 33, wherein the at least one preamplifier is connected directly to the non-invasive measuring electrode.

36. The apparatus according to claim 33, wherein the at least one preamplifier is connected to the non-invasive measuring electrode by a shielded cable.

37. The apparatus according to claim 36, wherein the shielded cable comprises a shield connected to an output of the at least one amplifying element.

38. The apparatus according to claim 27i wherein the at least one processing element comprises an analog/digital converter.
